(19) United States
(12) Patent Application Publication
(10) Pub. No.: US 2022/0245815 A1
(43) Pub. Date: Aug. 4, 2022

(54) APPARATUS AND METHOD FOR IDENTIFYING REAL-TIME BIOMETRIC IMAGE

(71) Applicant: XAIMED Co., Ltd., Seoul (KR)

(72) Inventor: Sang Min PARK, Seoul (KR)

(73) Assignee: XAIMED Co., Ltd., Seoul (KR)

(21) Appl. No.: 17/581,822

(22) Filed: Jan. 21, 2022

(30) Foreign Application Priority Data
Feb. 2, 2021 (KR) 10-2021-0014897

(51) Int. Cl. G06T 7/00; G06V 10/80

(52) U.S. Cl. CPC G06T 7/0016; G06T 2207/10088; G06T 2207/10081; G06V 10/806

(57) ABSTRACT

Provided are a computing device and methods for identifying real-time biometric image. In certain aspects, disclosed a method including the steps of: extracting a first feature information from a nth(n is a natural number) biometric image among biometric images continuously photographed temporally of an object based on a machine learning model; generating a fusion data using at least one sensor data among sensor data temporally corresponding to a n+1th or more biometric images and the first feature information of the nth biometric image; and extracting a second feature information of the n+1th or more biometric images from the fusion data based on a second machine learning model. This present disclosure application is a result developed through the Seoul Industry Promotion Agency's 2021 technology commercialization support project (TB210264), “Improvement and advancement of an explainable artificial intelligence prototype that detects major organs during laparoscopic surgery”.

### TECHNICAL FIELD

The present disclosure relates to identifying a real-time biometric image, more particularly, to an apparatus and methods for capable of predicting a location of an object by tracking the object in the real-time biometric image.

### DESCRIPTION OF THE RELATED ART

With development of artificial intelligence learning models, many machine learning models are being used to read medical images. For example, the machine learning models such as Convolutional Neural Networks (CNN), Deep Neural Networks (DNN), Recurrent Neural Networks (RNN), and Deep Belief Networks (DBN) are being applied to detect, classify, and characterize the medical images.

When real-time images (video images) are learned using a machine learning model, it is difficult to identify an object in a real-time image unless a performance of a processor or memory is high due to a large amount of learning and computation on image data.

As such, there is a need for an apparatus and methods capable of identifying the object in the real-time image based on the machine learning model regardless of the memory or processor performance.

This present disclosure application is a result developed through the Seoul Industry Promotion Agency's 2021 technology commercialization support project (TB210264), “Improvement and advancement of an explainable artificial intelligence prototype that detects major organs during laparoscopic surgery”.

### SUMMARY OF THE DISCLOSURE

In one aspect of the present disclosure, a computing device comprises a processor; and a memory that is communicatively coupled to the processor and stores one or more sequences of instructions, which when executed by the processor causes steps to be performed comprising: generating a fusion data using any one among biometric images continuously photographed temporally of an object and a sensor data corresponding in time to the biometric images; extracting a feature information of the biometric images from the fusion data based on a machine learning model.

Desirably, the feature information may include a label information of the object or a displacement information of the object for classifying the object identified in the biometric images.

Desirably, the displacement information may include at least one of a coordinate change amount, an angular change amount, an acceleration change amount, an angular acceleration change amount, a speed change amount, and an angular velocity change amount.

Desirably, the sensor data may be a data obtained from at least one of a gyro sensor, an acceleration sensor, and a magnetic sensor.

In another aspect of the present disclosure, computing device comprises a processor; and a memory that is communicatively coupled to the processor and stores one or more sequences of instructions, which when executed by the processor causes steps to be performed comprising: extracting a first feature information from a nth(n is a natural number) biometric image among biometric images continuously photographed temporally of an object based on a machine learning model; generating a fusion data using at least one sensor data among sensor data temporally corresponding to a n+1th or more biometric images and the first feature information of the nth biometric image; and extracting a second feature information of the n+1th or more biometric images from the fusion data based on a second machine learning model.

Desirably, the first machine learning model is different from the second machine learning model.

Desirably, the first feature information includes a label information for classifying the object which is identified in the nth biometric image.

Desirably, the second feature information includes a label information for classifying the object which is identified in the n+1th or more biometric images or a displacement information of the object.

### BRIEF DESCRIPTION OF THE DRAWINGS

References will be made to embodiments of the disclosure, examples of which may be illustrated in the accompanying figures. These figures are intended to be illustrative, not limiting. Although the disclosure is generally described in the context of these embodiments, it should be understood that it is not intended to limit the scope of the disclosure to these particular embodiments.

FIG. 1 shows a schematic diagram of an illustrative apparatus for identifying an object in a real-time biometric image according to embodiments of the present disclosure.

FIG. 2 is a schematic diagram of an illustrative system for identifying an object in a real-time biometric image according to embodiments of the present disclosure.

FIG. 3 shows a block diagram of a processor for identifying an object in a real-time biometric image according to embodiments of the present disclosure.

FIG. 4 shows a flowchart of an illustrative process for generating a feature information of a biometric image by a computing device according to one embodiment of the present disclosure.

FIG. 5 shows a flowchart of an illustrative process for generating a feature information of a biometric image by a computing device according to another embodiment of the present disclosure.

FIG. 6 is a view for explaining a process of displaying a biometric image using sensor data by a computing device according to one embodiment of the present disclosure.

FIG. 7 is a view for explaining a process of displaying a biometric image using sensor data by a computing device according to another embodiment of the present disclosure.

FIG. 8 shows a flowchart illustrating an exemplary process for identifying a real-time biometric image according to one embodiment of the present disclosure.

FIG. 9 shows a flowchart illustrating an exemplary process for identifying a real-time biometric image according to another embodiment of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity because of referring to performing machine learning by a processing module such as a processor, a CPU, an application processor, micro-controller, so on.

An “image” is defined as a reproduction or imitation of the form of a person or thing, or specific characteristics thereof, in digital form. An image can be, but is not limited to, a JPEG image, a PNG image, a GIF image, a TIFF image, or any other digital image format known in the art. “Image” is used interchangeably with “photograph”.

A “feature(s)” is defined as a group of one or more descriptive characteristics of subjects. A feature can be a numeric attribute.

The terms “comprise/include” used throughout the description and the claims and modifications thereof are not intended to exclude other technical features, additions, components, or operations.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

The embodiments described herein relate generally to diagnostic medical images. Although any type of medical image can be used, the disclosed methods, systems, apparatuses and devices can also be used with medical images of other ocular structures, or any other biological tissues image of which can support the diagnosis of a disease condition. Furthermore, the methods disclose herein can be used with a variety of imaging modalities including but not limited to: computed tomography (CT), magnetic resonance imaging (MRI), computed radiography, magnetic resonance, angioscopy, optical coherence tomography, color flow Doppler, cystoscopy, diaphanography, echocardiography, fluorescein angiography, laparoscopy, magnetic resonance angiography, positron emission tomography, single photon emission computed tomography, x - ray angiography, nuclear medicine, biomagnetic imaging, culposcopy, duplex Doppler, digital microscopy, endoscopy, fundoscopy, laser, surface scan, magnetic resonance spectroscopy, radio graphic imaging, thermography, and radio fluroscopy.

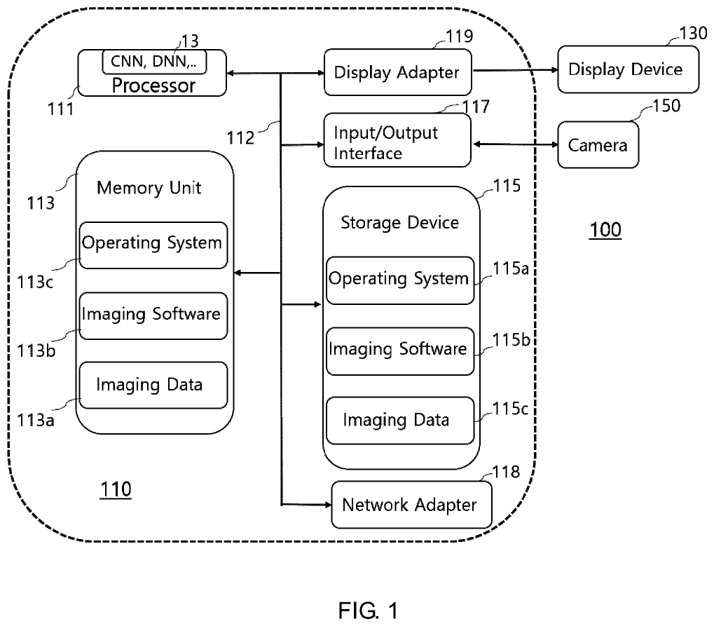

FIG. 1 shows a schematic diagram of an illustrative apparatus for identifying an object in a real-time biometric image according to embodiments of the present disclosure.

As depicted, the apparatus 100 may include a computing device 110, a display device 130 and a camera 150. In embodiments, the computing device 110 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 100 may further include communication mechanisms as well as the system bus 112 for transferring information. In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems.

In embodiments, the processor 111 is, but is not limited to, a processing module, a Computer Processing Unit (CPU), an Application Processor (AP), a microcontroller, a digital signal processor. In embodiments, the processor 111 may include an image filter such as a high pass filter or a low pass filter to filter a specific factor in the biometric image. In addition, in embodiments, the processor 111 may communicate with a hardware controller such as the display adapter 119 to display a user interface on the display device 130. In embodiments, the processor 111 may access the memory unit 113 and execute commands stored in the memory unit 113 or one or more sequences of instructions to control the operation of the apparatus 100. The commands or sequences of instructions may be read in the memory unit 113 from computer- readable medium or media such as a static storage or a disk drive, but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects(PCI), a PCI-Express bus, a Personal Computer Memory Card Industry Association (PCMCIA), Universal Serial Bus (USB) and the like. In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

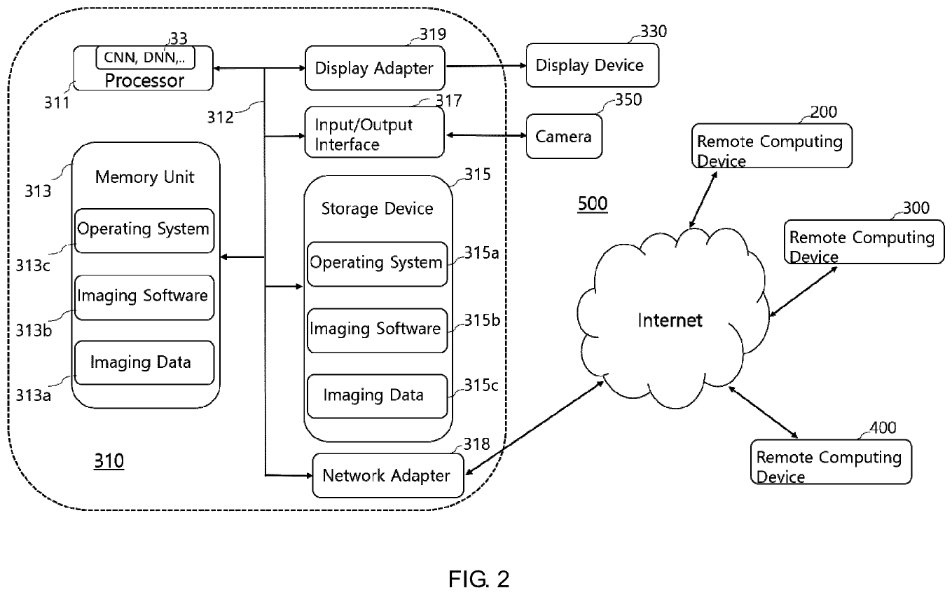

In embodiments, the apparatus 100 may transmit or receive the commands including messages, data, and one or more programs, i.e., a program code, through a network link or the network adapter 118. In embodiments, the network adapter 118 may include a separate or integrated antenna for enabling transmission and reception through the network link. The network adapter 118 may access a network and communicate with a remote computing devices 200, 300, 400 in FIG. 2.

In embodiments, the network may be, but is not limited to, at least one of

LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 110 may include a variety of computer-readable medium or media. The computer-readable medium or media may be any available medium or media that are accessible by the computing device 100. For example, the computer-readable medium or media may include, but is not limited to, both volatile and non-volatile media, removable or non-removable media.

In embodiments, the memory unit 113 may store a driver, an application program, data, and a database for operating the apparatus 100 therein. In addition, the memory unit 113 may include a computer-readable medium in a form of a volatile memory such as a random access memory (RAM), a non-volatile memory such as a read only memory (ROM), and a flash memory. For instance, it may be, but is not limited to, a hard disk drive, a solid state drive, an optical disk drive.

In embodiments, each of the memory unit 113 and the storage device 115 may be program modules such as the imaging software 113b, 115b and the operating systems 113c, 115c that can be immediately accessed so that a data such as the imaging data 113a, 115a is operated by the processor 111.

In embodiments, the machine learning model 13 may be installed into at least one of the processor 111, the memory unit 113 and the storage device 115. The machine learning model 13 may be, but is not limited to, at least one of a deep neural network (DNN), a convolutional neural network (CNN) and a recurrent neural network (RNN), which are one of the machine learning algorithms.

In embodiments, the camera 150 may include an image sensor (not shown) that captures an image of an object such as a liver, a pancreas, a gall bladder and photoelectrically converts the image into an image signal, and may photograph an image of the object using the image sensor. The photographed image may be stored in the memory unit 113 or the storage device 115, or may be provided to the processor 111 through the input/output interface 117 and processed based on the machine learning model 13. The photographed image may include a real-time image as well as a still image as a biometric image.

In addition, the camera 150 may include a sensor 151, various sensor data obtained by the sensor 151 when photographing a real-time biometric image of an object may be provided to the processor 111 through the input/output interface 117, and the various sensor data may be processed based on the machine learning model 13 together with the biometric image or may be stored in the memory unit 113 or the storage device 115. The sensor 151 may be a sensor that can obtain various data information, such as a position of the object (e.g., a tissue, an organ, etc.), a state of the object, and a direction of the object to be recognized in the biometric image, and may include, but is not limited thereto, a gyro sensor, an acceleration sensor, a magnetic sensor. The photographed real-time biometric image and the sensor data may be provided to remote computing devices 200, 300, and 400 to be described later through an Internet network.

FIG. 2 is a schematic diagram of an illustrative system for identifying an object in a real-time biometric image according to embodiments of the present disclosure.

As depicted, the system 500 may include a computing device 310 and one and more remote computing devices 200, 300, 400. In embodiments, the computing device 310 and the remote computing devices 200, 300, 400 may be connected to each other through a network. The components 310, 311, 312, 313, 315, 317, 318, 319, 330 of the system 500 are similar to their counterparts in FIG. 1. In embodiments, each of remote computing devices 200, 300, 400 may be similar to the apparatus 100 in FIG. 1. For instance, each of remote computing devices 200, 300, 400 may include each of the subsystems, including the processor 311, the memory unit 313, an operating system 313c, 315a, an imaging software 313b, 315b, an imaging data 313a, 315c, a network adapter 318, a storage device 315, an input/output interface 317 and a display adapter 319. Each of remote computing devices 200, 300, 400 may further include a display device 330 and a camera 350. In embodiments, the system bus 312 may connect the subsystems to each other.

In embodiments, the computing device 310 and the remote computing devices 200, 300, 400 may be configured to perform one or more of the methods, functions, and/or operations presented herein. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present invention may also be implemented into other computing devices and systems. Furthermore, aspects of the present invention may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

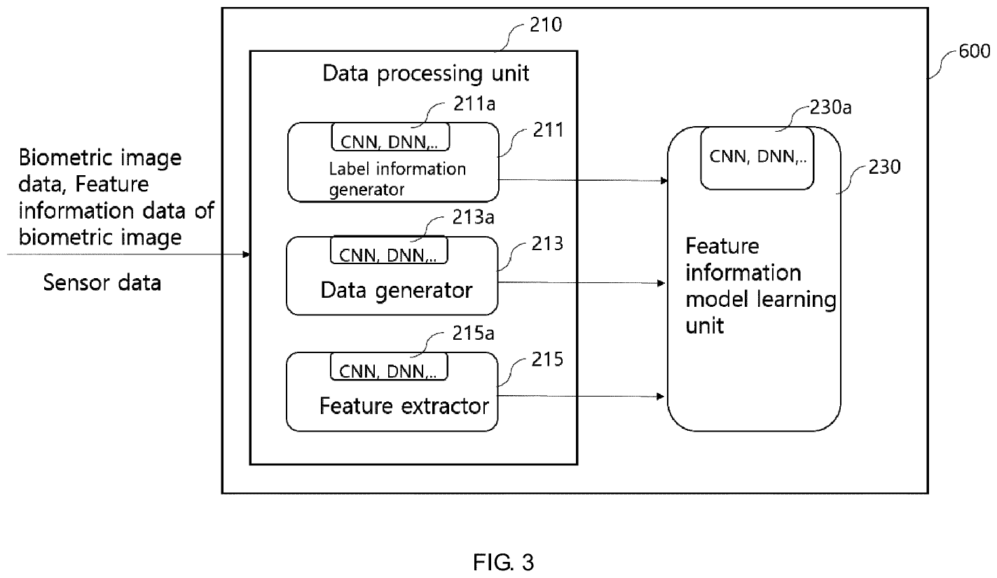

FIG. 3 shows a block diagram of a processor for identifying an object in a real-time biometric image according to embodiments of the present disclosure.

As depicted, in embodiments, the processor 600 may be the processor 111 and 311 shown in FIGS. 1 and 2. The processor 600 may receive a training data from the sensor 151, 351 or the camera 150, 350 to train the machine learning models 211a, 213a, 215a, and 230a, and may extract a feature information of the training data based on the received training data. The training data may be real-time biometric image data (a plurality of biometric image data or single biometric image data) or sensor data. Also, the training data may be feature information data or sensor data extracted from the real-time biometric image data. In embodiments, the feature information data may be label information for classifying a detected object in the biometric image data. For example, the label may be a category classified into internal organs such as liver, pancreas, and gallbladder expressed in the biometric image, and may be a category classified into internal tissues such as blood vessels, lymph, and nerves. In embodiments, the label information may include location information of the object, and the label may be given a weight or order based on the weight and meaning of the object recognized in the real-time biometric image data.

Also, the feature information may be displacement information in which an object recognized in current biometric image data is changed from an object recognized in previous biometric image data. For example, the feature information may be a feature vector indicating an angular change amount, an acceleration change amount, an angular acceleration change amount, a speed change amount, an angular velocity change amount, and the like of a recognized object. In this case, the feature vector may be extracted from the corresponding real-time biometric image data in conjunction with the sensor data.

The processor 600 may include a data processing unit 210 and a feature information model learning unit 230.

The data processing unit 210 may receive the real-time biometric image data and the sensor data or the feature information data of the real-time biometric image and the sensor data to train the feature information model. The data processing unit 210 may transform or process the received biometric image data and the sensor data, or the feature information data of the biometric image and the sensor data, into data suitable for training the feature information model. In embodiments, the data processing unit 210 may include a label information generator 211, a data generator 213, and a feature extractor 215.

The label information generator 211 may generate label information corresponding to the received real-time biometric image data using a first machine learning model 211a. The label information may be information on one or more categories according to an object recognized in the received real-time biometric image data. In embodiments, the label information may be stored in the memory unit 113 or the storage device 115 together with information on the real-time biometric image data corresponding to the label information.

The data generator 213 may generate data to input into the feature information model learning unit 230 including the machine learning model 230a. The data generator 213 may generate input data to input to the fourth machine learning model 230a based on a plurality of frame data included in the received real-time biometric image data using the second machine learning model 213a. The frame data may mean each frame constituting the real-time biometric image, may mean RGB data of each frame constituting the real-time biometric image, and may mean data extracting features of each frame or data in which features for each frame is expressed as a vector. In embodiments, the data generator unit 213 may generate the input data based on all of the plurality of frame data, and may generate regular input data based on any single frame data or odd-numbered or even-numbered frame data among the plurality of frame data. In addition, the data generator 213 may convert the received sensor data into data which is suitable for the feature information model.

The feature extractor 215 may extract a feature vector corresponding to the received real-time biometric image data using the third machine learning model 215a. For example, the feature extractor 215 may extract the feature vector representing a change in the position of the object recognized in the real-time biometric image data based on the sensor data.

The feature information model learning unit 230 includes the fourth machine learning model 230a. The data, which includes the image data, the label information and feature vectors, generated and extracted from each of the label information generator 211, the data generator 213, and the feature extractor 215 are input to the fourth machine learning model 230a. The feature information model learning unit 230 may extract feature information on the real-time biometric image data based on the data. The feature information refers to information related to the characteristics of the target image recognized in the real-time biometric image data. For example, the feature information may be the label (e.g., spleen) information that classifies objects in biometric image data, or the data that is possible to extract location information of an object using the sensor data sensing a movement of the camera within continuous biometric image data that can be obtained by photographing objects in the human body while the camera moves. If an error occurs in the feature information extracted by the feature information model learning unit 230, a coefficient or a connection weight value used in the fourth machine learning model 230a may be updated.

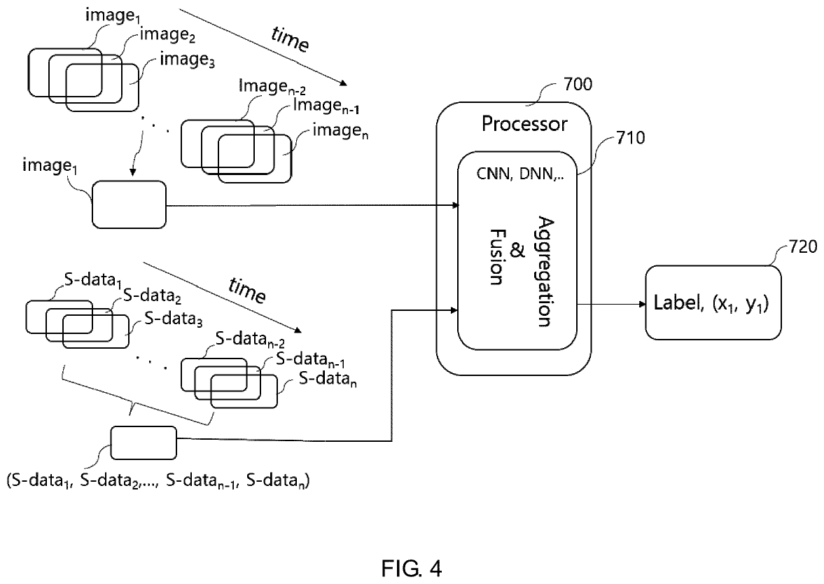

FIG. 4 shows a flowchart of an illustrative process for generating a feature information of a biometric image by a computing device according to one embodiment of the present disclosure.

As depicted, any one biometric image at any time among the continuous biometric images (image₁, image₂, . . . imageₙ₋, imageₙ) photographed in real time may be input to the machine learning model 710, and the sensor data (S-data₁, S-data₂. . . , S-dataₙ₋₁, S-dataₙ) temporally corresponding to the biometric images may be input to the machine learning model 710. The sensor data are values representing displacement of a biometric image obtained from sensors mounted on the camera according to a camera movement.

The processor 700 may extract a feature information 720 of any one biometric image after a certain point in time, by fusion & aggregation learning of one input biometric image and the sensor data based on the machine learning model 710 included therein. The processor 700 may be the processors 111 and 311 included in the computing devices 110 and 310 of the FIGS. 1 and 2.

In an embodiment, if an nth biometric image (e.g., image₁ with n=1) in temporal order among the continuously photographed biometric images is input to the machine learning model 710, and the sensor data (e.g., S-data₁, S-data₂, S-dataₙ₋₁, S-dataₙ, where n is 1 or more) temporally corresponding to the nth or more biometric images is input to the machine learning model 710, the processor 700 may extract the feature information 720 of the n+1th or more biometric images (e.g., image₂, imageₙ₋₁, imageₙ) by fusion learning the input biometric image and the sensor data based on the machine learning model 710.

In the case of extracting the feature information for a specific biometric image among the n+1th or more biometric images, for example, when extracting the feature information of only the fifth biometric image (image₅) in temporal order, the sensor data may utilize the first and fifth sensor data temporally corresponding to the first and fifth biometric images, instead of the first to fifth sensor data temporally corresponding to the first to fifth biometric images data, respectively.

The feature information 720 may be the label information or the displacement information of the object for classifying an object recognized in the extracted biometric image. As described above, the displacement information can be information that can know the position of an object in 2 dimension or 3 dimension, for example, numerical data that can be measured in conjunction with the biometric image such as two-dimensional or three-dimensional coordinates, rotation angle, angular velocity, angular acceleration, etc. The feature information 720 may be stored in the system memory unit 113 or the storage device 115 .

Although not shown, the machine learning model 710 may be input to a computer-readable recording medium and may be executed therein. The machine learning model 710 also may be input to the memory unit 113 or the storage device 115, and may be operated and executed by the processor 700 .

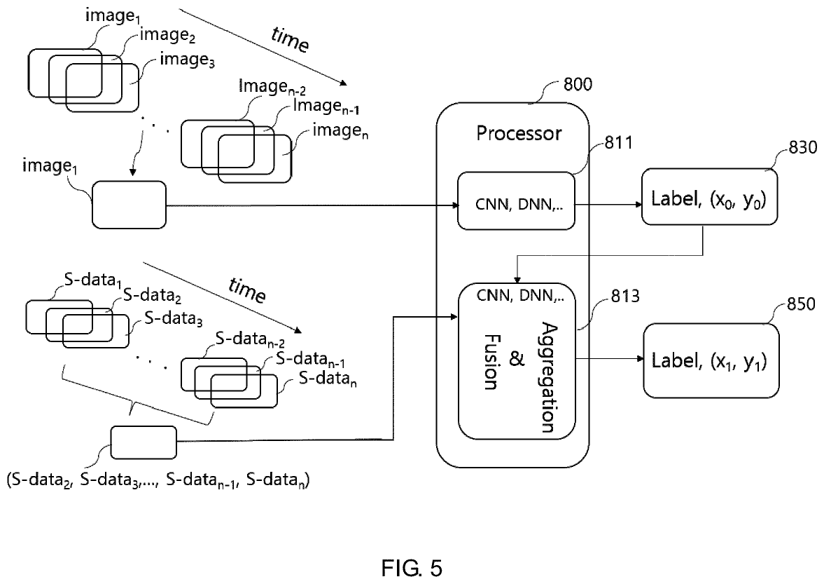

FIG. 5 shows a flowchart of an illustrative process for generating a feature information of a biometric image by a computing device according to another embodiment of the present disclosure.

As depicted, one biometric image at any time among the continuous biometric images (image₁, image₂, . . . . , imageₙ₋₁, imageₙ) photographed in real time may be input to a first machine learning model 811, the processor 800 may extract a first feature information 830 of the one input biometric image based on the first machine learning model 811. In embodiments, the processor 800 may be the processors 111 and 311 included in the computing devices 110 and 310 of the FIGS. 1 and 2. The first feature information 830 may be stored in the system memory unit 113 or the storage device 115. Then, the extracted first feature information 830 and the sensor data (S-data₂, . . . , S-dataₙ₋₁, S-dataₙ) temporally corresponding to the continuous biometric images after the any time may be input to a second machine learning model 813. The sensor data are values representing displacement of a biometric image according to camera movement from sensors mounted on the camera. The processor 800 may extract a second feature information 720 of any one biometric image after a certain point in time, by fusion & aggregation learning of the first feature information 830 of the one input biometric image and the sensor data based on the second machine learning model 813 included therein.

In an embodiment, if an nth biometric image (e.g., image₁ with n=1) in temporal order among the continuously photographed biometric images is input to the first machine learning model 811, the processor 800 may extract the first feature information 830 of the n-th biometric image (image₁) based on the first machine learning model 811. In this case, the first feature information 830 may be the label information for classifying an object recognized in the biometric image and the location information ((x₀, y₀)) of the object. Then, if the first feature information 830 of the extracted n-th biometric image (image₁) and the sensor data (e.g., S-data₂, S-dataₙ₋₁, S-dataₙ) temporally corresponding to the nth or more biometric images is input to the second machine learning model 813, the processor 800 may extract a second feature information 850 of the n+1th or more biometric images (e.g., image₂, imageₙ₋₁, imageₛ) by fusion learning the first feature information and the sensor data based on the second machine learning model 813.

The second feature information 850 may be the label information or the displacement information of the object for classifying an object recognized in the extracted biometric image. The displacement information of the object may be expressed as location information ((x₁, y₁)). Also, the displacement information can be information that can know the position of an object in 2 dimension or 3 dimension, for example, numerical data that can be measured in conjunction with the biometric image such as two-dimensional or three-dimensional coordinates, rotation angle, angular velocity, angular acceleration, etc. The second feature information 850 may be stored in the system memory unit 113 or the storage device 115 .

In one embodiment, the first machine learning model 811 and the second machine learning model 813 may have the same neural network configuration such as the number of layers, the number of nodes in each layer, and connection settings between nodes of adjacent layers. In another embodiment, the first machine learning model 811 and the second machine learning model 813 are different machine learning models. For example, the first machine learning model 811 may be composed of a neural network for extracting position information for recognizing the coordinates of the object and the label information for classifying objects in biometric images, and the second machine learning model 813 may be composed of a neural network for extracting displacement information for recognizing the displacement such as position, angle, and rotation of the object as well as the label information and the coordinates of the object for classifying the objects in the biometric image.

The first machine learning model 811 and the second machine learning model 813 may be input to a computer-readable recording medium and may be executed therein. The first machine learning model 811 and the second machine learning model 813 also may be input to the memory unit 113 or the storage device 115, and may be operated and executed by the processor 800.

Thus, when utilizing the sensor data that can be measured in real time according to the movement of the camera, even if the present computing device does not extract the feature information by processing all of the continuous biometric images based on the machine learning model, it can extract the feature information from continuous biometric images in real time. Accordingly, it is possible to reduce the amount of computation for all of the continuous biometric images, thereby supplementing the performance of the processor.

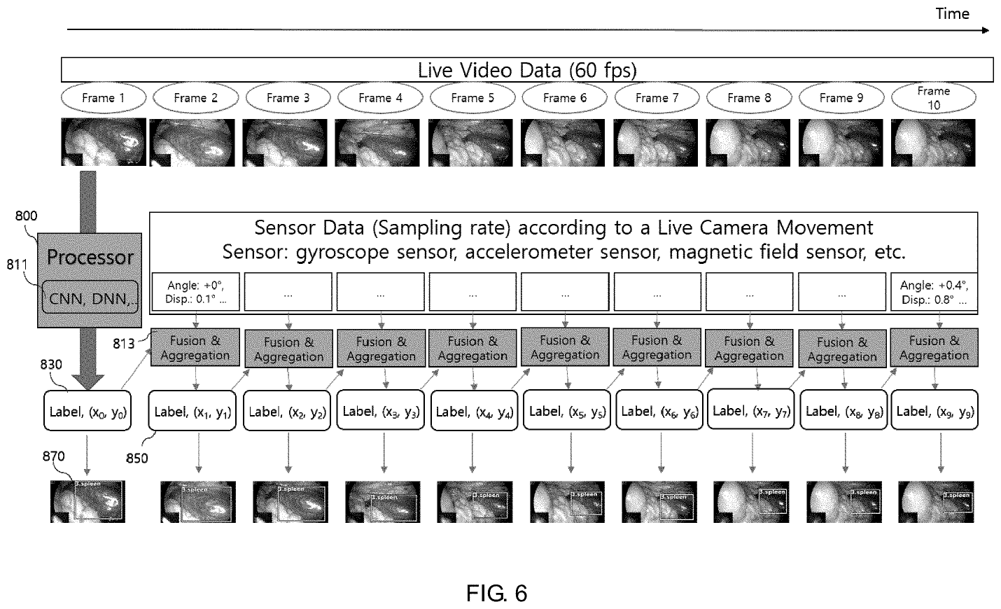

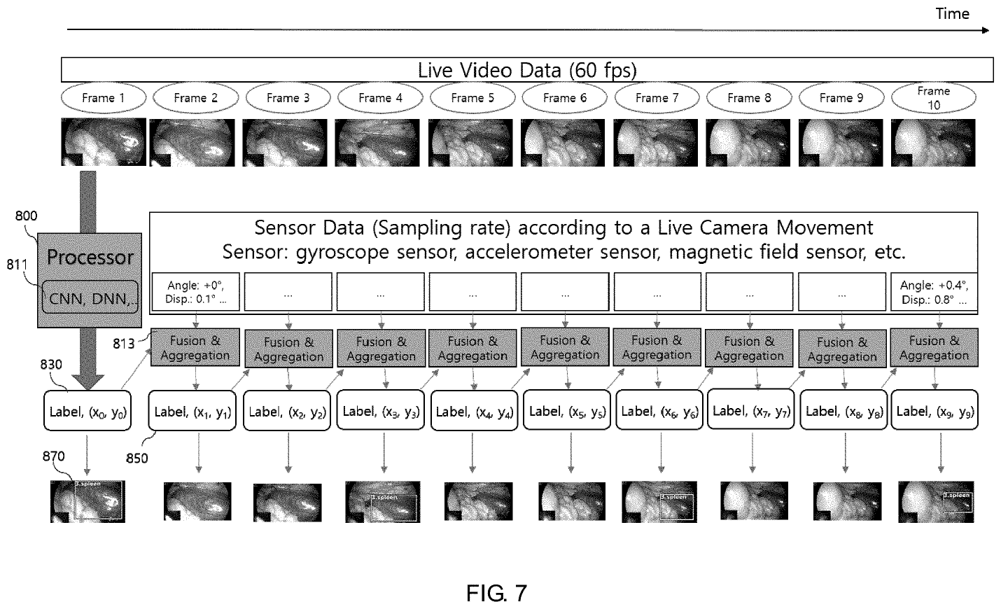

FIG. 6 is a view for explaining a process of displaying a biometric image using sensor data by a computing device according to one embodiment of the present disclosure, FIG. 7 is a view for explaining a process of displaying a biometric image using sensor data by a computing device according to another embodiment of the present disclosure.

As depicted, all feature information 830 and 850 of continuous biometric images(Frame1, Frame2, Frame3, . . . , Frame10) photographed in real time may be extracted by the above-described method in conjunction with FIG. 5. As shown in FIG. 6, the extracted feature information may be image-processed by the processor 800 to generate the labeled biometric images 870 temporally corresponding to all the consecutive biometric images, and the labeled biometric images 870 may be displayed on the devices 130 and 330. Also as shown in FIG. 7, the extracted feature information may be image-processed by the processor 800 only for any biometric images among all the consecutive biometric images, the labeled biometric images temporally corresponding to the any biometric images may be generated through image processing by the processor 800 and may be displayed on a display device.

As such, if a device for identifying the biometric image according to the present disclosure generates the labeled biometric image by using the sensor data, the position of a visually invisible object (e.g., organ, tissue) in the biometric image can be predicted.

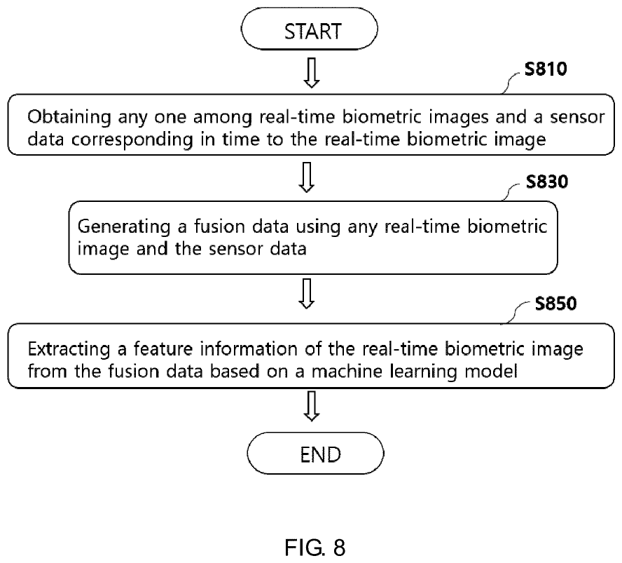

FIG. 8 shows a flowchart illustrating an exemplary process for identifying a real-time biometric image according to one embodiment of the present disclosure.

Referring to FIG. 8, any one of the photographed biometric images and sensor data may be used to extract feature information of the photographed biometric images in real time. At step S810, the processor 700 described in conjunction with FIG. 4 may obtain any biometric image among real-time biometric images photographed for the object and sensor data from a sensor capable of measuring temporally changing biometric image information while simultaneously photographing the biometric image. The sensor data is corresponding to the biological images in time. In embodiments, the real-time biometric image may be a streaming image that captures an organ or tissue inside the human body in real time using a camera such as a flexible endoscope or a laparoscopic endoscope. In particular, the real-time biometric image may be any biometric image obtained by photographing the inside of the human body in real time during surgery. Anything can apply. In embodiments, the sensor data may be data obtained from various sensors capable of measuring a change of the photographed biometric image according to the movement of a camera.

At step S830, fusion data may be generated by the processor 700 using the any biometric images and the sensor data. The fusion data may be generated based on a machine learning model, and may be generated by a processor capable of complex processing images and data. The fusion data may be obtained by concatenating the features (or feature vectors) of the any biometric image and the features (feature vectors) of the sensor data, or by summing the features (feature vectors) of the any biometric image and the features of the sensor data.

Thereafter, at step S850, the processor 700 may extract the feature information of all of the real-time biometric images from the fusion data based on a machine learning model. In embodiments, the machine learning model may be composed of a neural network for extracting displacement information for recognizing the displacement such as position, angle, and rotation of the object as well as the label information and the coordinates of the object for classifying the objects in the biometric image. For example, the machine learning model may be a deep neural network (DNN), a convolutional neural network (CNN), a recurrent neural network (RNN), etc., which are one of the machine learning algorithms.

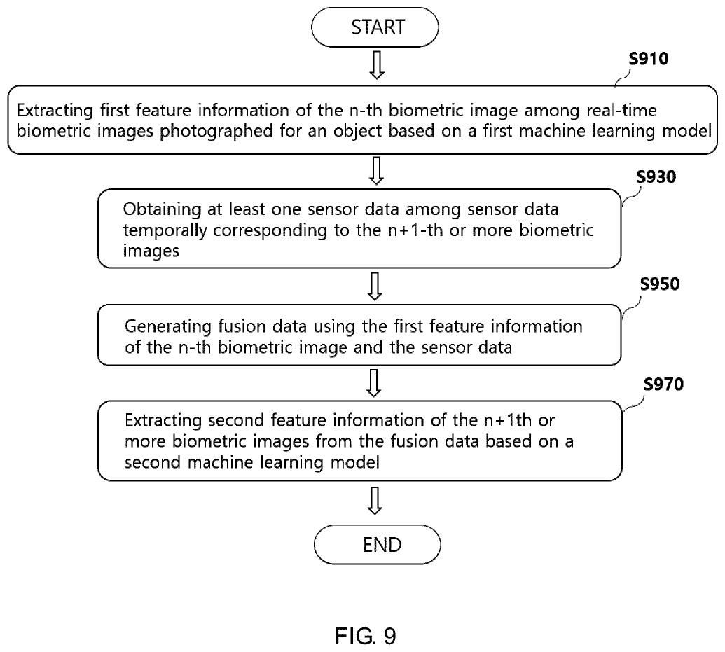

FIG. 9 shows a flowchart illustrating an exemplary process for identifying a real-time biometric image according to another embodiment of the present disclosure.

Referring to FIG. 9, feature information of any one of the photographed biometric images and sensor data may be used to extract the feature information of the photographed biometric images in real time. At step S910, the processor 800 described in conjunction with FIG. 5 may extract first feature information of the n-th biometric image among real-time biometric images photographed for an object based on a first machine learning model. the first machine learning model may be composed of a neural network for extracting position information for recognizing the coordinates of the object and the label information for classifying objects in biometric images. In embodiments, the real-time biometric image may be a streaming image that captures an organ or tissue inside the human body in real time using a camera such as a flexible endoscope or a laparoscopic endoscope.

At step S930, the processor 800 may obtain at least one sensor data among sensor data temporally corresponding to the n+1-th or more biometric images. The sensor data may be data obtained from various sensors capable of measuring a change of the photographed biometric image according to the movement of a camera.

At step S950, fusion data may be generated by the processor 800 using the first feature information of the n-th biometric image and the sensor data. In embodiments, the fusion data may be generated based on a machine learning model, and may be generated by a processor capable of complex processing images and data. In addition, the fusion data may be obtained by concatenating the features (or feature vectors) of the any biometric image and the features (feature vectors) of the sensor data, or by summing the features (feature vectors) of the any biometric image and the features of the sensor data.

In step S970 , the processor 800 may extract second feature information of the n+1th or more biometric images from the fusion data based on a second machine learning model. The second machine learning model may be composed of a neural network for extracting displacement information for recognizing the displacement such as position, angle, and rotation of the object as well as the label information and the coordinates of the object for classifying the objects in the biometric image.

The extraction of feature information of these real-time biometric images may be performed by a computing device, which is a device that receives a real-time photographed biometric image dataset as training data, and may generate data learned as a result of performing the machine learning model. In the disclosure of each operation belonging to the method according to the present embodiment, if the description of the subject is omitted, it may be understood that the subject of the corresponding operation is the computing device or the processor.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. A computing device comprising:
a processor; and
a memory that is communicatively coupled to the processor and stores one or more sequences of instructions, which when executed by the processor causes steps to be performed comprising:
generating a fusion data using any one among biometric images continuously photographed temporally of an object and a sensor data corresponding in time to the biometric images;
extracting a feature information of the biometric images from the fusion data based on a machine learning model.

2. The computing device of claim 1,
wherein the feature information includes a label information of the object or a displacement information of the object for classifying the object identified in the biometric images.

3. The computing device of claim 2,
wherein the displacement information includes at least one of a coordinate change amount, an angular change amount, an acceleration change amount, an angular acceleration change amount, a speed change amount, and an angular velocity change amount.

4. The computing device of claim 1,
wherein the sensor data is a data obtained from at least one of a gyro sensor, an acceleration sensor, and a magnetic sensor.

5. A computing device comprising:
a processor; and
a memory that is communicatively coupled to the processor and stores one or more sequences of instructions, which when executed by the processor causes steps to be performed comprising:
extracting a first feature information from a nth(n is a natural number) biometric image among biometric images continuously photographed temporally of an object based on a machine learning model;
generating a fusion data using at least one sensor data among sensor data temporally corresponding to a n+1th or more biometric images and the first feature information of the nth biometric image; and
extracting a second feature information of the n+1th or more biometric images from the fusion data based on a second machine learning model.

6. The computing device of claim 5,
wherein the first machine learning model is different from the second machine learning model.

7. The computing device of claim 5,
wherein the first feature information includes a label information for classifying the object which is identified in the nth biometric image.

8. The computing device of claim 5,
wherein the second feature information includes a label information for classifying the object which is identified in the n+lth or more biometric images or a displacement information of the object.

9. A method for identifying real-time biometric image, comprising:
extracting a first feature information from a nth(n is a natural number) biometric image among biometric images continuously photographed temporally of an object based on a machine learning model;
generating a fusion data using at least one sensor data among sensor data temporally corresponding to a n+1th or more biometric images and the first feature information of the nth biometric image; and
extracting a second feature information of the n+1th or more biometric images from the fusion data based on a second machine learning model.

10. The method of claim 9,
wherein the first machine learning model is different from the second machine learning model.

11. The method of claim 9,
wherein the first feature information includes a label information for classifying the object which is identified in the nth biometric image.

12. The method of claim 9,
wherein the second feature information includes a label information for classifying the object which is identified in the n+1th or more biometric images or a displacement information of the object.
